(19) United States
(12) Patent Application Publication
(10) Pub. No.: US 2017/0303892 A1
(43) Pub. Date: Oct. 26, 2017

(54) TRANSDUCER ORIENTATION MARKER

(71) Applicant: B-K Medical Aps, Herlev (DK)

(72) Inventors: John J. ANTOL, Nahant, MA (US); Niels-Christian SASADY, Frederiksberg (DK); Henrik JENSEN, Bagsvaerd (DK)

(73) Assignee: B-K Medical Aps, Herlev (DK)

(21) Appl. No.: 15/513,216

(22) PCT Filed: Sep. 24, 2014
(86) PCT No.: PCT/IB2014/002532

§ 371 Date: Mar. 22, 2017

(51) Int. Cl. A61B 8/00; G01S 7/52; G01S 15/89

(52) U.S. Cl. CPC A61B 8/4254; G01S 15/8915; G01S 7/52084; A61B 8/4455; A61B 8/463; A61B 8/4483; A61B 8/54

(57) ABSTRACT

An imaging system comprises an ultrasounds probe (102) including a housing (108) with a probe orientation marker (116) disposed on the housing. The imaging system further comprises a display (132). The imaging system further comprises a console (104), electrically interfaced with the probe and the display, that includes a controller (128). The controller is configured to visually present an ultrasound image, in electronic format and via the display, with an image orientation marker visually displayed superimposed over the image and selectively located with respect to the displayed image based on the location of the probe orientation marker on the housing.

### TECHNICAL FIELD

The following generally relates to ultrasound imaging and more particularly to an ultrasound imaging transducer orientation marker.

### BACKGROUND

An ultrasound imaging system has included an ultrasound probe and a console. The console includes a processor and memory, application software, a communication interface connector, etc. and interfaces with a display monitor and a user interface. The ultrasound probe includes a housing, a transducer array housed by the housing, and cable with connector. The probe and console communicate through the connectors.

The transducer array includes transducing elements that transmit an ultrasound signal in response to being excited and that sense echoes produced in response to the signal interacting with structure. In B-mode, the echoes are processed, producing a sequence of focused, coherent echo samples along focused scanlines of a scanplane. The scanlines are scan converted into a format of a display monitor and visually presented as image via the display monitor.

The probe housing has included a small fin near one side of the transducer array that protrudes out from the housing. The fin indicates a left/right orientation of the transducer array. By convention, the fin should point toward the patient's right side in transverse views and head in longitudinal views. The displayed image has been overlaid with an on-screen marking that corresponds to the fin. The side of the image corresponding with the fin end of the transducer is shown onscreen with a colored orientation marker. In this manner, the sonographer will be visually apprised of the image plane and orientation of the displayed image.

To add an orientation fin to a probe that does not already have an orientation fin, the mold tooling would need to be changed to include the orientation fin, which would add cost. For probes with two part handles, the orientation fin should be at the dividing line, which requires extra care. For probes that already have an orientation fin, the fin may make it more difficult to clean the probe as it may require extra time and/or additional cleaning attention around and at the fin.

### SUMMARY

Aspects of the application address the above matters, and others.

In one aspect, an imaging system comprises an ultrasounds probe including a housing with a probe orientation marker disposed on the housing. The imaging system further comprises a display. The imaging system further comprises a console, electrically interfaced with the probe and the display, which includes a controller. The controller is configured to visually present an ultrasound image, in electronic format and via the display, with an image orientation marker visually displayed superimposed over the image and selectively located with respect to the displayed image based on the location of the probe orientation marker on the housing.

In another aspect, a method includes receiving a signal indicating an orientation of an ultrasound probe. The method further includes identifying, with a processor, the orientation of the ultrasound probe from the signal. The method further includes visually displaying, via a display, an ultrasound image generated with data detected by an array of transducer elements of the ultrasound probe. The method further includes overlaying, with the processor, a graphic over the displayed image, wherein the graphic mimics a graphic on the probe and indicates an image plane and orientation of the displayed image with respect to an image plane and orientation of the ultrasound probe.

In another aspect, an ultrasound probe including a transducer array with a plurality of transducer elements, a housing that supports the transducer array, wherein the housing includes an outer surface and a probe orientation marker embedded in the outer surface. The probe orientation marker identifies an imaging plane and a spatial orientation of the transducer array within the ultrasounds probe.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates an example ultrasound imaging system;

FIG. 2 illustrates a first side of an example probe;

FIG. 3 illustrates a second side of the example probe of FIG. 2 with a probe orientation marker;

FIG. 4 illustrates the second side of the example probe of FIG. 3 in connection with sub-windows displaying an image and an image orientation marker for different combinations of displayed image plane and orientation;

FIG. 5 illustrates a variation of FIG. 4 in which the probe orientation marker and the mage orientation marker include a graphic of a company logo;

FIG. 6 illustrates a first side of another example probe;

FIG. 7 illustrates a second side of the other example probe of FIG. 6 with the probe orientation marker disposed thereon;

FIG. 8 illustrates a first side of another example probe;

FIG. 9 illustrates a second side of the other example probe of FIG. 8 with the probe orientation marker disposed thereon;

FIG. 10 illustrates a first side of another example probe;

FIG. 11 illustrates a second side of the other example probe of FIG. 10 with the probe orientation marker disposed thereon;

FIG. 12 illustrates an example of the ultrasound imaging system; and

FIG. 13 illustrates an example method in accordance with the description herein.

### DETAILED DESCRIPTION

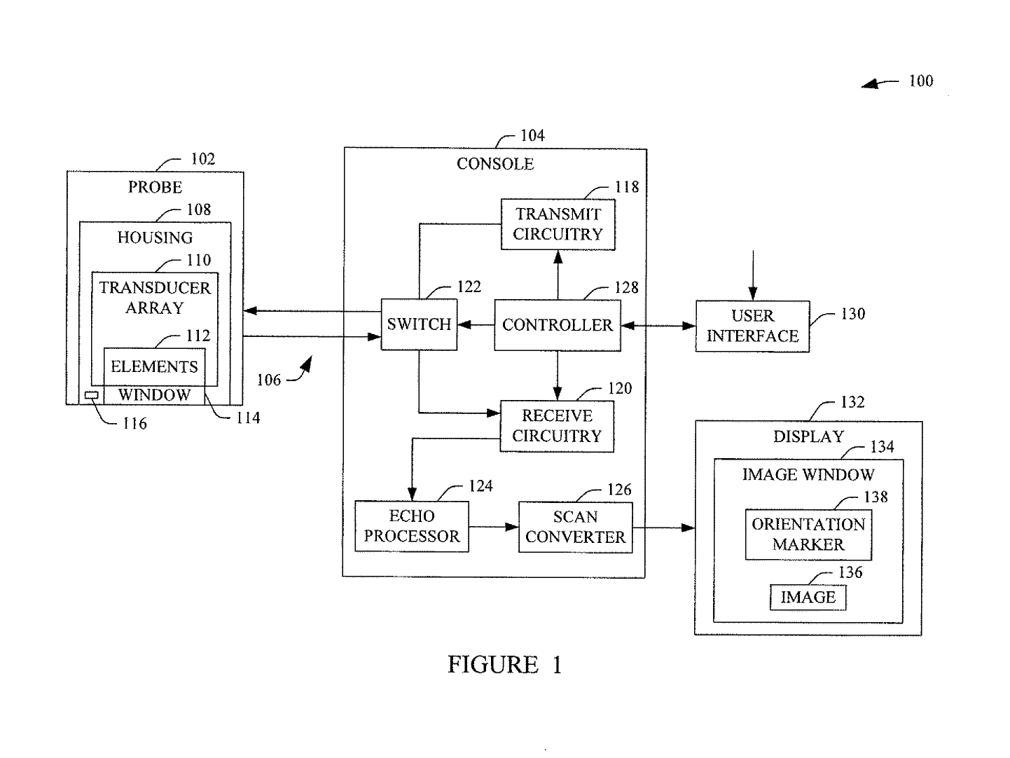

FIG. 1 illustrates an example imaging system 100, such as an ultrasound imaging system.

The imaging system 100 includes a probe 102 and a console 104, which are configured to interface over a communications path 106. In one instance, the communications path 106 is through an electrical-mechanical connection of complementary connectors of the probe 102 and the console 104. In another instance, the communications path 106 is through respective wireless interfaces.

The probe 102 includes a housing 108, a transducer array 110 of transducer elements 112, and an acoustic window 114. The transducer array 110 can include one or more rows of the transducer elements 112, which are configured to transmit ultrasound signals and receive echo signals. Suitable arrays 110 include linear, curved, and/or otherwise shaped. The transducer array 110 can be fully populated or sparse.

The housing 108 houses or encloses the transducer array 110, which is mechanically supported by and/or within the housing 108. The acoustic window 114 is disposed in and/or about a material free region on a side of the housing 108 next to a transducing side of the transducer elements 112 and serves as an interface between the transducer array 110 and the surrounding environment.

The illustrated housing 108 has a probe orientation marker 116. As described in greater detail below, the probe orientation marker 116 is disposed at a predetermined location on the housing 108 and visually indicates information such as the image plane and/or an orientation of the probe 102. For example, in one instance, the probe orientation marker 116, with respect to the probe 102, indicates whether the probe 102 is facing up or down, left or right, etc.

The console 104 includes transmit circuitry 118 configured to generate a set of radio frequency (RF) pulses that are conveyed to the transducer array 110 and selectively excite a set of the transducer elements 112, causing the elements to transmit ultrasound signals. The console 104 further includes receive circuitry 120 that senses or receives echoes (RF signals) generated in response to the transmitted ultrasound signals interacting with structure (e.g., organ cells, blood cells, etc.).

The console 104 further includes a switch 122. The switch 122 switches between the transmit circuitry 118 and the receive circuitry 120, depending on whether the transducer array 110 is being operated in transmit mode or receive mode. In transmit mode, the switch 122 electrically connects the transmit circuitry 118 to the transducer elements 112. In receive mode, the switch 122 electrically connects the receive circuitry 120 to the transducer elements 112.

The console 104 further includes an echo processor 124 that processes received echoes. Such processing may include applying time delays, weighting on the channels, summing, and/or otherwise beamforming received echoes. In B-mode, the echo processor 124 produces a sequence of focused, coherent echo samples along focused scanlines of a scanplane. Other processing may lower speckle, improve specular reflector delineation, and/or includes FIR filtering, IIR filtering, etc.

The console 104 further includes a scan converter 126. The scan converter 126 scan converts the output of the echo processor 124 creating images for display. The console 104 further includes a controller 128. The controller 128 controls one or more of the transmit circuitry 118, the receive circuitry 118, the switch 122, the echo processor 124 and/or the scan converter 126. Such control can be based on available modes of operation. Examples of such modes of operation include one or more of B-mode, Doppler mode, etc.

In the illustrated embodiment, the console 104 also interfaces a user interface (UI) 130 and a display 132. In another embodiment, at least one of the user interface 130 or the display 132 are integrated in and part of the console 104. The UI 130 may include one or more input devices (e.g., buttons, knobs, trackball, etc.) and/or one or more output devices (e.g., visual, audio, etc. indicators). The UI 130 can be used to select an imaging mode, etc.

The display 132 includes an image window 134 configured to display an image, such as an ultrasound image 136, and an image orientation marker 138, which indicates an orientation of the displayed image. As described in greater detail below, in one non-limiting instance, the image orientation marker 138 includes a graphic, which mirrors the probe orientation marker 116 in that the graphic visually resembles or looks the same as the probe orientation marker 116. The image orientation marker 138 is overlaid over the image based on a predetermined location so at to indicate an orientation of the displayed image with respect to the current actual physical orientation of the probe 102 with respect to the subject or object being scanned.

It is to be appreciated that the console 104 includes one or more processor (e.g., a central processing unit, a microprocessor, etc., and memory or computer readable medium which excludes transitory and includes physical memory) encoded with computer executable instructions. The instructions, when executed by the processor, cause the processor to perform one or more of the functions described herein.

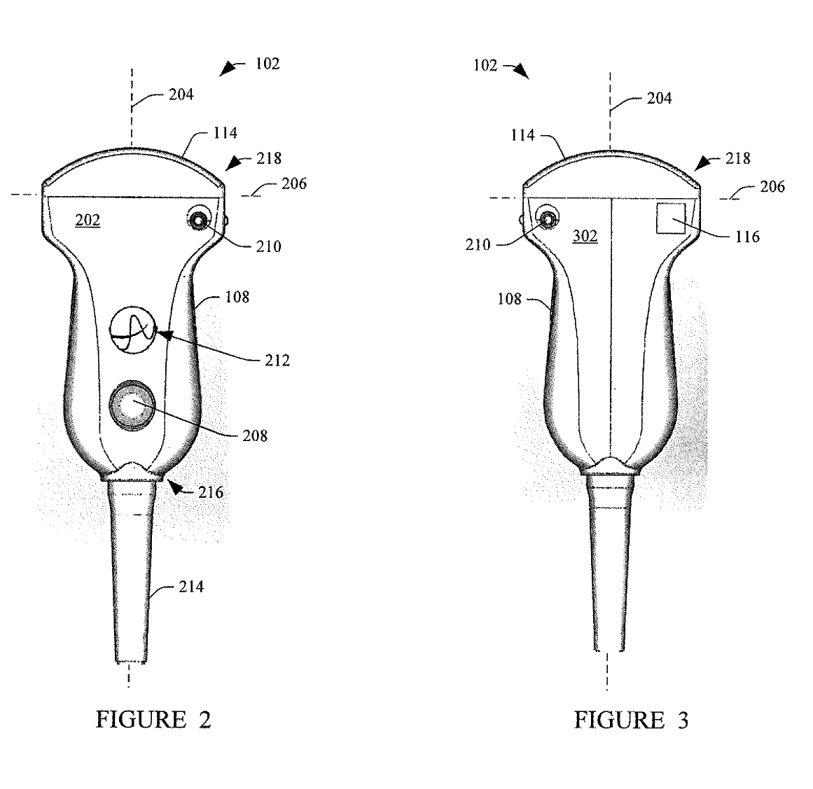

FIGS. 2 and 3 depict an example of the probe 102. FIG. 2 shows a top side 202 of the probe 102, and FIG. 3 shows a bottom side 302 of the probe 102. The top side 202 of the probe 102 and the bottom side 302 of the probe 102 are opposing sides of the probe 102.

With reference to FIGS. 2 and 3, the probe 102 has a long axis 204 and a short axis 206, which is transverse to the long axis 204. The transducer elements 112, which are not visible in FIGS. 2 and 3 since they are located within the housing 108 and behind the acoustic window 114, extend sequentially along the short axis 206 in the azimuth direction.

The illustrated top side 202 (FIG. 2) includes at least one user control 208, which is integrated in the top side 202. For example, the user control 208 can be a physical button extending from inside of the housing 108, through an opening in the housing 108, and protruding out from the housing 108. In another instance, the user control 208 can be part of a touch sensitive surface of the housing 108. The illustrated housing 108 further includes a puncture guide 210.

The illustrated housing 108 further includes a graphic 212 (FIG. 2 only). In the illustrated example, the graphic 212 includes a logo. The probe 102 also includes a cable 214 which routes electrical channels between the individual transducer elements 112 and the connector of the cable. The cable 214 extends from a back 216 of the probe 102, which is opposite a probe head 218, which includes the acoustic window 114 and the transducer elements 112.

The bottom side 302 (FIG. 3) includes the probe orientation marker 116. The probe orientation marker 116 is disposed in an orientation in which it is non-inverted when the probe is in scanning mode, e.g., meaning the array 110 pointing down at the object and the cable 214 pointing up away from the object. The probe orientation marker 116 is disposed proximate to the probe head 218 and outer peripheral transducer elements of the transducer array 110, symmetrically disposed with respect to puncture guide 210 about the long axis 204, and aligned with respect to puncture guide 210 about the long axis 204.

The illustrated probe orientation marker 116 occupies a square footprint. A size of the illustrated probe orientation marker 116 is a size on an order of 2.5 to 7.5±0.05 millimeters (mm), such as 4.8±0.05 mm, 4 9±0.05 mm, 5 0±0.05 mm, 5.1±0.05 mm, or 5.2±0.05 mm, or other size in the range of 2.5 to 7.5±0.05 mm. In another embodiment, the probe orientation marker 116 can be smaller or larger and/or occupy a rectangular, circular, oval, irregular, etc. footprint.

In one instance, the probe orientation marker 116 is an engravement in the housing 108. For example, the probe orientation marker 116 can be laser and/or otherwise engraved in the housing 108. Generally, an engravement is easier to clean relative to a protrusion type (e.g., a fin) physical marker. In another instance, the probe orientation marker 116 can be tattooed, painted on, and/or otherwise placed on the housing 108. The probe orientation marker 116 may or may not include color.

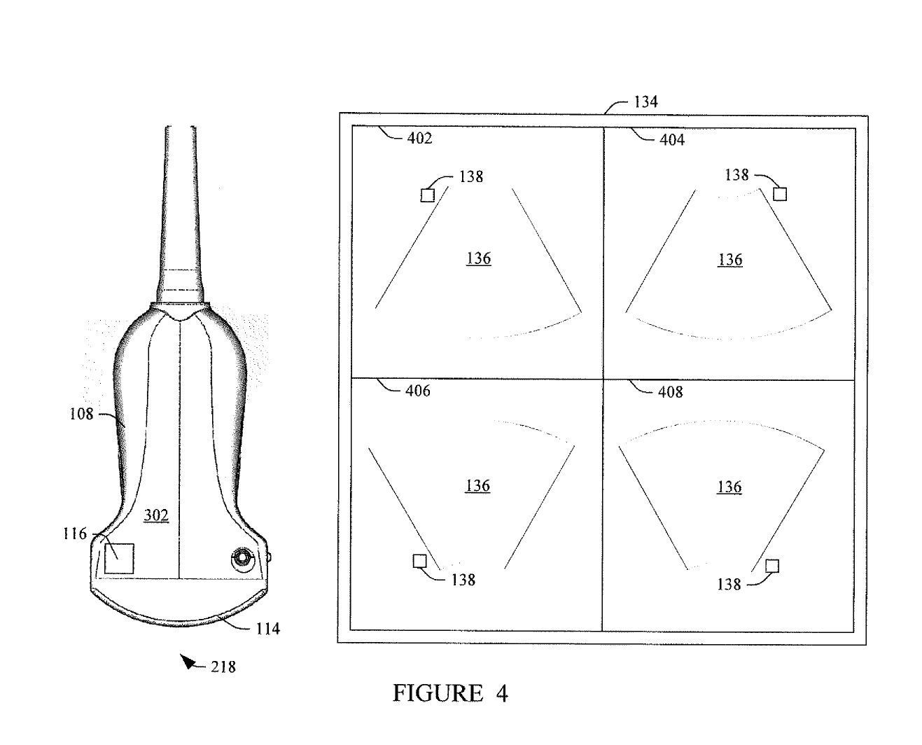

FIG. 4 depicts the probe 102 of FIGS. 2 and 3 in connection with the image window 134 of the display 132. In this example, a plurality of sub-windows 402, 404, 406 and 408 are displayed in the image window 134, each with a different image plane and orientation.

The sub-window 402 shows an image plane and orientation corresponding to the orientation of the illustrated probe 102. That is, the probe head 218 faces down and the probe orientation marker is on the left side of the probe. As such, the image plane is shown facing down and the image orientation marker 138 is on the upper left side of the displayed image.

In the sub-window 404, the orientation has been rotated 180 degrees (e.g., from left to right), and the image orientation marker 138 is on the upper right side of the displayed image (e.g., moved from left to right). The image plane remains facing down. Alternatively, this represents the orientation of the probe 102 after rotating the probe 102 180 degrees while keeping the probe head 218 facing down.

In the sub-window 406, the image plane has been rotated 180 degrees (e.g., from down to up), and the image orientation marker 138 is on the lower right side of the displayed image. The image plane faces up. Alternatively, this represents the orientation of the probe 102 after rotating the probe 102 180 degrees to face up while keeping the orientation the same.

In the sub-window 408, both the image plane and the orientation have been rotated 180 degrees. In this example, the image orientation marker 138 is on the lower right side of the displayed image and the image plane faces up. Alternatively, this represents the orientation of the probe 102 after rotating the probe 102 180 degrees to face up and rotating the probe head 218.

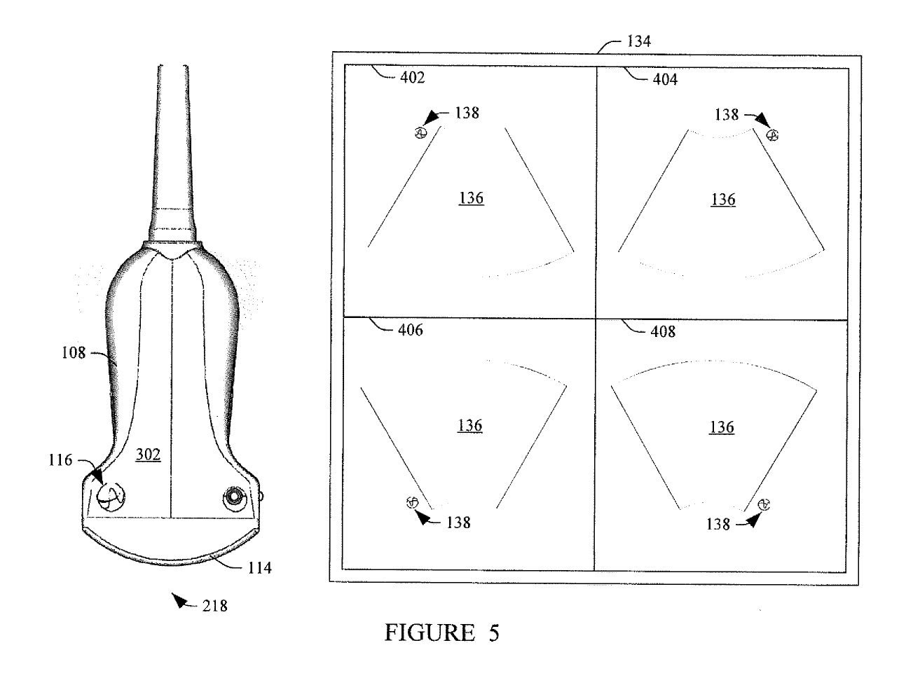

FIG. 5 depicts the example described in connection with FIG. 4 in which both the probe orientation marker 116 and the image orientation marker 138 are the same graphic. In this example, the graphic is a log such as a company logo. In another embodiment, at least one of the probe orientation marker 116 or the image orientation marker 138 is a different graphic.

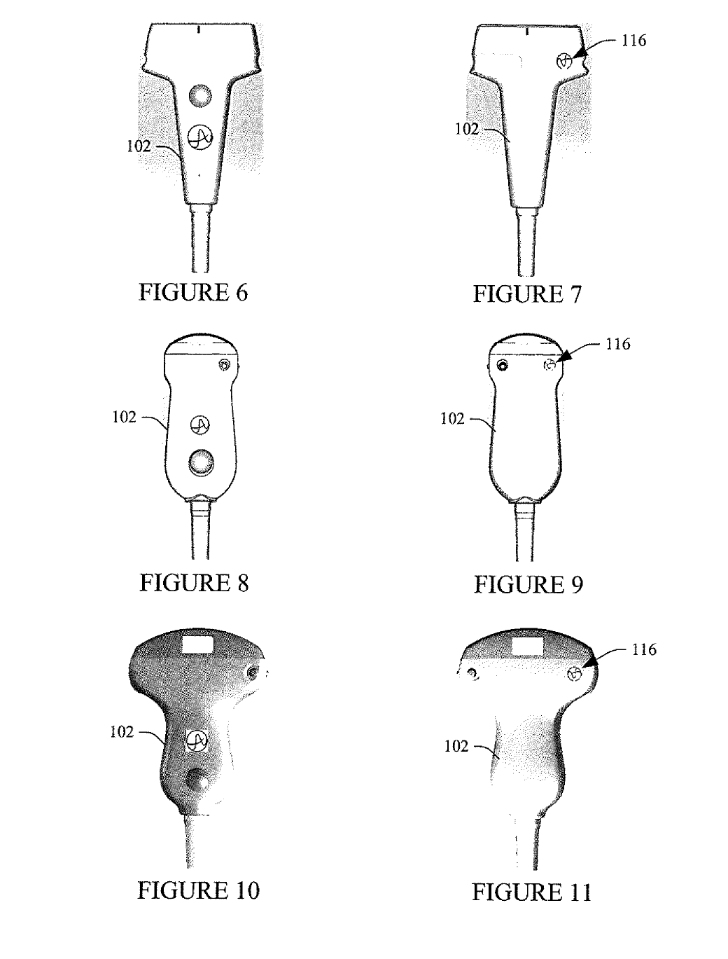

FIGS. 6, 7, 8, 9, 10 and 11 depict example of other probes 102 with the probe orientation markers 116. FIGS. 6 and 7 depict top and bottom views of the same probe. FIGS. 8 and 9 depict top and bottom views of the same probe. FIGS. 10 and 11 depict top and bottom views of the same probe. In a variation, the probe orientation marker 116 can additionally or alternatively be disposed on the top side 202. In another variation, different colors can be used to indicate the image plane and orientation instead of mirroring and/or inverting the probe orientation marker 116.

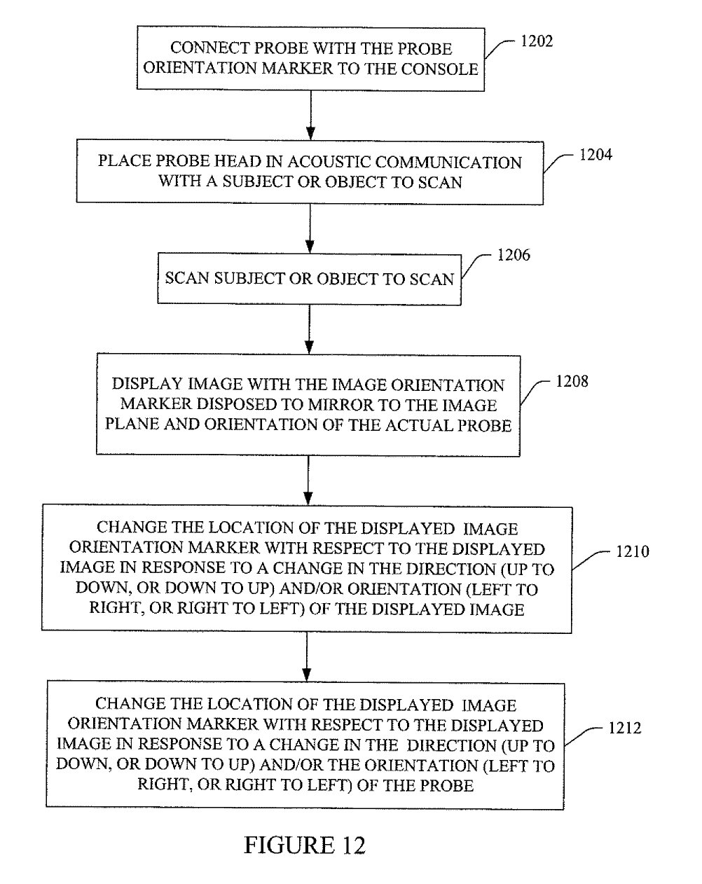

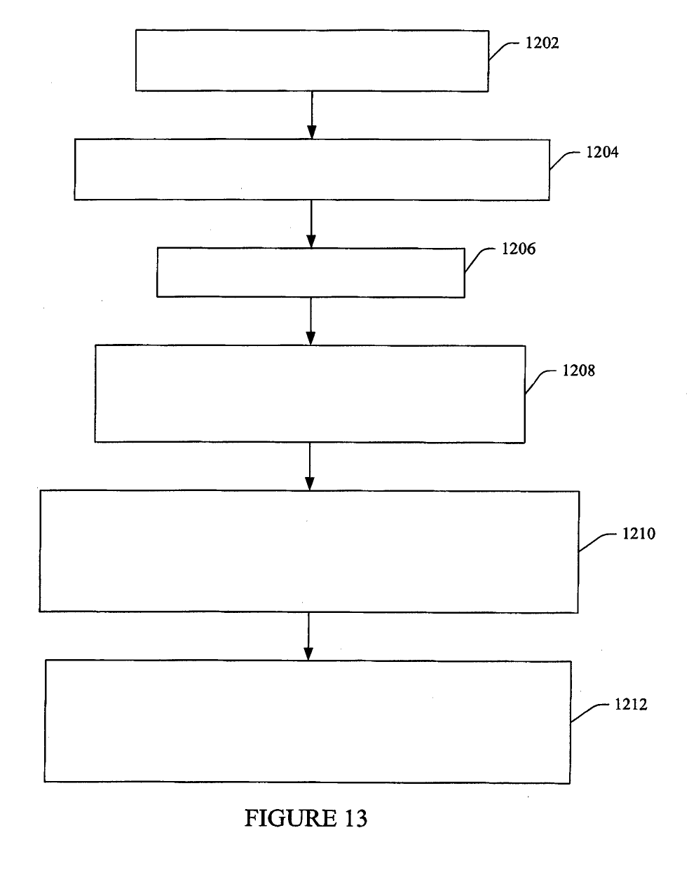

FIG. 12 illustrates a non-limiting example of the ultrasound imaging system 100. In this example, the console 104 is affixed to a mobile cart 1204, which include movers 1206 such as wheels, casters, etc., the user interface 130 is part of console 104, and the display 132 is affixed to the mobile cart 1204. In another configuration, the ultrasound imaging system 100 does not include movers, but instead is configured to rest on a table, desk, etc.

FIG. 12 illustrates a method.

It is to be appreciated that the order of the following acts is provided for explanatory purposes and is not limiting. As such, one or more of the following acts may occur in a different order. Furthermore, one or more of the following acts may be omitted and/or one or more additional acts may be added.

At 1202, the probe 102 is connected to the console 104.

At 1204, the probe head 218 of the probe is placed in acoustic communication with a subject or object to be scanned.

At 1206, the subject or object is scanned.

At 1208, an image generated in response to the scan is displayed with the image orientation marker 138 superimposed thereover to mirror the image plane and orientation of the actual probe 102.

At 1210, the location of the displayed image orientation marker 138 is changed in response to switching the display direction and/or orientation. In a variation, the act is omitted.

At 1212, the location of the image orientation marker 138 is changed in response to changing the physical direction and/or orientation of the probe. In a variation, the act is omitted.

At least a portion of the method discussed herein may be implemented by way of computer readable instructions, encoded or embedded on computer readable storage medium (which excludes transitory medium), which, when executed by a computer processor(s), causes the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. An imaging system, comprising:
an ultrasounds probe including a housing with a probe orientation marker disposed on the housing;
a display; and
a console, electrically interfaced with the probe and the display, that includes a controller configured to visually present an ultrasound image, in electronic format and via the display, with an image orientation marker visually displayed superimposed over the image and selectively located with respect to the displayed image based on the location of the probe orientation marker on the housing.

2. The imaging system of claim 1, wherein the probe orientation marker identifies a spatial orientation of the ultrasounds probe with respect to a subject or object to scan.

3. The imaging system of claim 1, wherein the probe orientation marker includes a first graphic, the image orientation marker includes a second graphic, and the first graphic and the second graphic are a same graphic.

4. The imaging system of claim 3, wherein the same graphic is a company logo.

5. The imaging system of claim 1, wherein the controller visually presents the image orientation marker to mirror the spatial orientation of the ultrasound probe.

6. The imaging system of claim 1, wherein the controller visually presents the image orientation marker based on an image plane of the ultrasounds probe.

7. The imaging system of claim 6, wherein the controller visually presents the image orientation marker to mirror to the image plane of the ultrasounds probe with respect to a subject or object to scan.

8. The imaging system of claim 1, wherein the ultrasounds probe includes a transducer array of elements, and the probe orientation marker is disposed on an outside of the housing proximate to a region of the probe 102 housing an end region of the transducer array.

9. The imaging system of claim 1, wherein the housing includes a long axis and the probe orientation marker is disposed on one side of the long axis.

10. The imaging system of claim 1, wherein the probe orientation marker is disposed within a region having a size on an order of five millimeters.

11. The imaging system of claim 1, wherein the probe orientation marker is an engravement in the housing.

12. A method, further comprising:
receiving a signal indicating an orientation of an ultrasound probe;
identifying, with a processor, the orientation of the ultrasound probe from the signal;
visually displaying, via a display, an ultrasound image generated with data detected by an array of transducer elements of the ultrasound probe; and
overlaying, with the processor, a graphic over the displayed image, wherein the graphic mimics a graphic on the probe and indicates an image plane and orientation of the displayed image with respect to an image plane and orientation of the ultrasound probe.

13. The method of claim 12, wherein the graphic is a company logo.

14. The method of claim 12, wherein the graphic is an engravement.

15. The method of claim 12, further further comprising:
receiving a second signal indicating a change in a direction of the displayed image plane;
visually displaying that ultrasound image based on the second signal; and
positioning the graphic over the displayed image based on the change.

16. The method of a claim 12, further comprising:
receiving a second signal indicating a change in an orientation the displayed image;
visually displaying that ultrasound image based on the second signal; and
positioning the graphic over the displayed image based on the change.

17. The method of claim 12, further comprising:
receiving a second signal indicating a change in a direction of the displayed image plane and a change in an orientation the displayed image;
visually displaying that ultrasound image based on the second signal; and
positioning the graphic over the displayed image based on the change.

18. The method of claim 12, further comprising:
receiving a third signal indicating a change in a direction of the image plane of the probe;
visually displaying that ultrasound image based on the third signal; and
positioning the graphic over the displayed image based on the change so that the graphic mimics the graphic on the probe and indicates the image plane of the displayed image with respect to the image plane of the ultrasound probe.

19. The method of claim 12, further comprising:
receiving a third signal indicating a change in an orientation of the probe;
visually displaying that ultrasound image based on the third signal; and
positioning the graphic over the displayed image based on the change so that the graphic mimics the graphic on the probe and indicates the orientation of the displayed image with respect to the orientation of the ultrasound probe.

20. The method of claim 12, further comprising:
receiving a third signal indicating a change in a direction of the image plane of the probe and in an orientation of the probe;
visually displaying that ultrasound image based on the third signal; and
positioning the graphic over the displayed image based on the change so that the graphic mimics the graphic on the probe and indicates the image plane and the orientation of the displayed image with respect to the image plane and the orientation of the ultrasound probe.

21. An ultrasounds probe, comprising:
a transducer array with a plurality of transducer elements;
a housing that supports the transducer array, wherein the housing includes an outer surface; and
a probe orientation marker embedded in the outer surface,
wherein the probe orientation marker identifies an imaging plane and a spatial orientation of the transducer array within the ultrasounds probe.

22. The ultrasounds probe of claim 21, wherein the probe orientation marker includes a company logo.
